(19) United States
(12) Patent Application Publication
(10) Pub. No.: US 2024/0180206 A1
(43) Pub. Date: Jun. 6, 2024

(54) FLAVOR COMPOSITIONS FOR BEVERAGE AND PERSONAL CARE APPLICATIONS

(71) Applicant: Firmenich SA, Satigny (CH)

(72) Inventors: Qiu-Min MA, Shanghai (CN); Lei SHI, Shanghai (CN); Fan ZHANG, Shanghai (CN)

(73) Assignee: Firmenich SA, Satigny (CH)

(21) Appl. No.: 18/550,934

(22) PCT Filed: Mar. 23, 2022
(86) PCT No.: PCT/EP2022/057607

§ 371 Date: Sep. 15, 2023

(30) Foreign Application Priority Data
Mar. 25, 2021 (WO) PCT/CN2021/082995
May 7, 2021 (EP) 21172630.2

(51) Int. Cl. A23L 2/56; A23L 2/68; A23L 27/00; A61K 8/06; A61K 8/73

(52) U.S. Cl. CPC A23L 2/56; A23L 2/68; A23L 27/80; A61K 8/068; A61K 8/73; A61K 2800/52; A61K 2800/596

(57) ABSTRACT

The present invention relates to a flavor delivery system comprising a flavor composition comprising a surfactant system comprising lysolecithin, lecithin, a sugar ester, a monoglycerol ester, a polyglycerol ester, or mixtures thereof, a non-polar phase comprising a flavor oil, a polar phase comprising at least one polar non-aqueous solvent, wherein the flavor delivery system includes a stabilizer system comprising a biopolymer. The invention further relates to a method for preparing a flavor delivery system, a beverage or personal care product comprising the flavor delivery system, a method for preparing a flavored beverage or personal care product, and the use of a biopolymer for modifying, increasing or enhancing the storage stability of a flavor composition in an acidic beverage or consumer product.

### TECHNICAL FIELD

The present invention relates to a flavor delivery system comprising a flavor composition comprising a surfactant system comprising lysolecithin, lecithin, a sugar ester, a monoglycerol ester, a polyglycerol ester, or mixtures thereof, a non-polar phase comprising a flavor oil, a polar phase comprising at least one polar non-aqueous solvent, wherein the flavor delivery system includes a stabilizer system comprising a biopolymer. The invention further relates to a method for preparing a flavor delivery system, a beverage or personal care product comprising the flavor delivery system, a method for preparing a flavored beverage or personal care product, and the use of a biopolymer for modifying, increasing or enhancing the storage stability of a flavor composition in an acidic beverage or consumer product.

### BACKGROUND OF THE INVENTION

For beverage and personal care applications, flavor compositions are desired.

Flavor compositions can be in the form of emulsions comprising a polar phase, an oil phase, as well as a surfactant system. The aqueous phase typically comprises water and/or one or more polar co-solvents and further ingredients. The oil phase is commonly dispersed within the aqueous phase thereby forming an oil-in-water emulsion. The dispersed oil phase typically comprises flavor oil(s) and optionally further lipophilic ingredients.

Surfactants are needed in the flavor compositions in order to prepare stable emulsified flavor compositions, as many flavor oils suitable for incorporation in beverages or consumer products have limited solubility in polar solvents, such as water.

Many different compounds have been proposed as surfactants for flavor compositions, such as sugar esters, lecithin, lysolecithin, monoglycerol esters, and polygylycerol esters of fatty acids. These compounds are acceptable for a wide range of beverage applications. However, when used in acid beverage applications, these surfactants show only limited stability, i.e. they do either show no stability at acidic pH-levels at all, or only for a very limited duration. This particularly applies to highly acidic beverages with a pH-value of around 3 or below. A lack of stability often manifests itself in that the acidic beverages are not clear in appearance anymore shortly after the addition of a flavor composition. By contrast, the beverages are rather cloudy in appearance due to the formation of undesirable precipitates.

As most of the popular beverages are acidic beverages with rather low pH-values, there is a need to provide solutions that allow the use of well-established surfactants also at rather low pH-values in order to ensure stability of these acidic beverages over a prolonged period (e.g. one month at 40° C. or 6-9 months at room temperature). Prolonged stability of these beverages also allows the storage and handling of these acidic beverages for a longer period.

The present invention provides solution for the above-mentioned objectives.

In particular, the present invention concerns a flavor delivery system that comprises a flavor composition comprising well-established surfactants, wherein the flavor delivery system includes a stabilizer system comprising a biopolymer.

The biopolymer that is included in the flavor delivery system can provide the flavor composition with increased stability at acidic conditions, e.g. when the flavor composition is used for acidic beverage applications. The stabilizer system thus allows the use of a flavor composition comprising well-established surfactants also under acidic conditions, as still sufficient stability over a prolonged period can be achieved.

### DETAILED DESCRIPTION OF THE INVENTION

The present invention relates to a flavor delivery system comprising
a flavor composition comprising
   a surfactant system comprising lysolecithin, lecithin, a sugar ester, a monoglycerol ester, a polyglycerol ester, or mixtures thereof,
   a non-polar phase comprising a flavor oil,
   a polar phase comprising at least one polar non-aqueous solvent;
wherein the flavor delivery system includes a stabilizer system comprising a biopolymer.

In a particular embodiment, the flavor delivery system is an emulsion. An emulsion is to be understood as a mixture of two lipids that are immiscible due to their different polarities (hydrophobic vs. hydrophilic). In an emulsion, one liquid (dispersed or internal phase) is dispersed in another liquid (external or continuous phase). Therefore, the non-polar phase may be dispersed within the polar-phase, or the polar phase may be dispersed within the non-polar phase.

In a particular embodiment, the non-polar phase is dispersed within the polar phase. In another particular embodiment, the polar phase is dispersed within the non-polar phase. Preferably, the non-polar phase is dispersed within the polar phase.

In a particular embodiment, the emulsion is a microemulsion. Microemulsions including micellar solutions are usually transparent dispersions that form spontaneously without the need of energy input when the compounds thereof are properly mixed with each other. In view of the small size of dispersed oil-droplets, typically being in a range of between 10 and 300 nm, the visible light cannot be scattered and thus microemulsions appear as clear or translucent isotropic solutions. A typical oil-in-water microemulsion consists of water, a co-solvent such as an alcohol, oil, and one or more surfactants and co-surfactants. A high proportion of oil is feasible for microemulsion systems, which saves transportation and storage costs. Microemulsions are also thermodynamically stable.

In another particular embodiment, the emulsion is a nanoemulsion. In contrast to microemulsions, nanoemulsions are usually prepared by high-energy input, such as high-pressure homogenization to break the big droplets into small ones. The size of the oil-droplets is typically between 5 and 200 nm.

The flavor composition according to the present invention comprises a surfactant system comprising lysolecithin, lecithin, a sugar ester, a monoglycerol ester, a polyglycerol ester, or mixtures thereof.

A surfactant system is required to obtain a composition that is thermodynamically stable at least for a considerable period. Surfactants show amphiphilic properties meaning that they contain both hydrophobic and hydrophilic moieties. Based on these structural properties, surfactants are surface-active, which allows them to reduce the interfacial tension between a polar and non-polar phase and thus, to stabilize an emulsion.

The lysolecithin that can be used is not particularly restricted. For example, use can be made of enzyme-modified lecithin, enzyme-treated lecithin or enzyme-hydrolyzed lecithin which contains as the main component lysolecithin (1-monoacylglycerophospholipid), which is obtained by enzymatically hydrolyzing natural substance-derived lecithin (1,2-diacylglycerophospholipid) including, but not limited to lecithin from soybeans, eggs, sunflower or rapeseed (canola) seeds, milk, marine sources, and cottonseeds, and then eliminating the thus formed free fatty acids and fat-soluble components originating in the starting materials. The enzyme to be used herein is not particularly restricted and exemplified by phospholipase, lipase, etc.

In a particular embodiment, the lysolecithin is one member selected from the group consisting of lysophosphatidylcholine, lysophosphatidylethanolamine, lysophosphatidylinositol, lysophosphatidylgylcerol, lysophosphatidylserine, or any mixture thereof.

Under “lecithin” a mixture of phospholipids is understood. Phospholipids in lecithin include positively charged phospholipids (e.g. phosphatidylcholine, phosphatidylethanolamine, and phosphatidylserine) and negatively charged phospholipids (e.g. phosphatidic acid, phosphatidylglycerol, and phosphatidylinositol). Lecithin is prepared by extracting and purifying phospholipids from naturally occurring products including, but not limited to, soybeans, eggs, sunflower or rapeseed (canola) seeds, milk, marine sources, and cottonseeds. Food-grade lecithin is available in liquid, granular and powdered form from commercial sources. The term “lecithin” as used herein refers to both a single type of lecithin as well as to a mixture of different types of lecithin.

In a particular embodiment, the lecithin is obtained from sunflower. Preferably, the lecithin is Lipoid Lecithin H100.

In a particular embodiment, lecithin is obtained from soybean. Preferably, the lecithin is Lipoid P 45 or Lipoid P 20.

In a particular embodiment, the sugar ester is a sugar ester of fatty acids. Preferably, the fatty acids are selected from the group consisting of palmitic acid, myristic acid, lauric acid, and stearic acid.

In a particular embodiment, the sugar ester is a sucrose ester of fatty acids, or a mixture of sucrose esters of fatty acids. Preferably, the fatty acids are selected from the group consisting of palmitic acid, myristic acid, lauric acid, and stearic acid. Preferably, the sugar ester comprises sucrose monostearate or sucrose monopalmitate.

In a particular embodiment, the monoglycerol ester is an ester of fatty acids. Preferably, the fatty acids in the monoglycerol ester have a carbon number of from 12 to 18, preferably the fatty acids are stearic acid, oleic acid or a combination thereof.

In a particular embodiment, the polyglycerol ester is an ester of fatty acids. Preferably, the fatty acids in the polyglycerol esters ester have a carbon number of from 12 to 18, preferably the fatty acids are stearic acid, oleic acid or a combination thereof

In a particular embodiment, the polyglycerol ester has an average degree of polymerization of from 3 to 10 glycerol units.

In a particular embodiment, the surfactant system comprises lysolecithin.

In a particular embodiment, the flavor delivery system comprises lysolecithin in an amount of from 10 to 40 wt. %, based on the total weight of the flavor delivery system, preferably in an amount of 15 to 35 wt. %, more preferably in an amount of 19 to 28 wt. %.

In a particular embodiment, the surfactant system consists of lysolecithin.

In a particular embodiment, the surfactant system comprises lecithin.

25 In a particular embodiment, the flavor delivery system comprises lecithin in an amount of 1 to 10 wt. %, based on the total weight of the flavor delivery system, preferably in an amount of 2 to 9 wt. %, more preferably in an amount of 3 to 5 wt. %.

In a particular embodiment, the surfactant system consists of lecithin.

In a particular embodiment, the surfactant system comprises lysolecithin and a sugar ester, or lysolecithin and lecithin.

In a particular embodiment, the surfactant system comprises a sugar ester.

In a particular embodiment, the flavor delivery system comprises a sugar ester in an amount of 1 to 20 wt. %, based on the total weight of the flavor delivery system, preferably in an amount of 3 to 12 wt. %, more preferably in an amount of 7 to 12 wt. %.

In a particular embodiment, the surfactant system consists of a sugar ester.

In a particular embodiment, the surfactant system comprises a sugar ester and lecithin. The flavor composition further comprises a non-polar phase comprising a flavor oil.

Under “non-polar phase” is to be understood the total amount of hydrophobic compounds in the flavor composition.

In a particular embodiment, the non-polar phase may further include one or more other active ingredients selected from oil-soluble pharmaceutical ingredients, oil-soluble nutraceutical ingredients (e.g., oil-soluble vitamins), oil-soluble colorants, oil-soluble antimicrobial ingredients, oil-soluble defoamers, mouthfeel modulators, taste modulators, and any combinations thereof. Useful taste modulators include acid maskers, polyaldo matric, beer hops, cooling agents, hot tasting substances, sweet enhancers, salt enhancers, salivation-inducing substances, substances causing a warmth or tingling feeling, and any combinations thereof. Exemplary mouthfeel modulators are coconut oil, coconut milk with or without sugar, vanillin, medium chain triglycerides, and combinations thereof. At least one cooling compound is selected from the group consisting of: 2-(4-ethylphenoxy)-N-(IH-pyrazol-5-yl)-N-(2-thienylmethyl)acetamide, WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (IR,2S,5R)-N-(4-Methoxyphenyl)-p-menthanecarboxamide, FEMA 4681; WS-27 (N-Ethyl-2,2-diisopropylbutanamide), FEMA 4557; N-Cyclopropyl-5-methyl-2-isopropylcyclohexanecarboxamide, FEMA 4693, WS-116 (N-(I,I-Dimethyl-2-hydroxyethyl)-2,2-diethylbutanamide), N-(I,I-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (IR,2S,5R)-N-(4-Methoxyphenyl)-p-menthanecarboxamide (WS-12), FEMA 4681; (2S,5R)-N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy]ethanol, FEMA 4718; (2,6-Diethyl-5-isopropyl-2-methyltetrahydropyran, FEMA 4680); trans-4-tert-Butylcyclohexanol, FEMA 4724; 2-(p-tolyloxy)-N-(IH-pyrazol-5-yl)-N-((thiophen-2-yl)methyl)acetamide, FEMA 4809; Menthone glycerol ketal, FEMA 3807; Menthone glycerol ketal, FEMA 3748; (-)-Menthoxypropane-1,2-diol; 3-(I-Menthoxy)-2-methylpropane-1,2-diol, FEMA 3849; Isopulegol; (+)-cis & (−)-trans p-Menthane-3,8-diol, Ratio ˜62:38, FEMA 4053; 2,3-dihydroxy-p-menthane; 3,3,5-trimethylcyclohexanone glycerol ketal; menthyl pyrrolidone carboxylate; (IR,3R,4S)-3-menthyl-3,6-dioxaheptanoate; (IR,2S,5R)-3-menthyl methoxyacetate; (IR,2S,5R)-3-menthyl 3,6,9-trioxadecanoate; (IR,2S,5R)-3-menthyl 3.6,9-trioxadecanoate; (IR,2S,5R)-3-menthyl (2-hydroxyethoxy) acetate; (IR,2S,5R)-menthyl I I-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N-(R)-2-oxotetrahydrofuran-3-yl-(IR,2S,5R)-p-menthane-3-carboxamide; mixture of 2,2,5,6,6-pentamethyl-2,3,6,6a-tetrahydropentalen-3a(IH)-ol and 5-(2-hydroxy-2-methylpropyl)-3,4,4-trimethylcyclopent-2-en-I-one; (IR,2S,5R)- 2-isopropyl-5-methyl-N-(2-(pyridin-2-yl)ethyl) cyclohexanecarboxamide, FEMA 4549; (2S,5R)- 2-isopropyl-5-methyl-N-(2-(pyridin-4-yl)ethyl)cyclohexanecarboxamide; N-(4-cyanomethylphenyl) p-menthanecarboxamide, FEMA 4496; (IS,2S,5R)-N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-4-cyano-N-phenyl-N-[2-(pyridin-2-yl)ethyl]benzenesulfonamide; yl)ethyl]benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4-((9H-fluoren-9-ylamino)methyl)-2-methoxyphenol; 4-((benzhydrylamino)methyl)-2-ethoxyphenol; I-(4-methoxyphenyl)-2-(I-methyl-1H-benzo[d]imidazol-2-yl)vinyl4-methoxybenzoate; 2-(I-isopropyl-6-methyl-IH-benzo[d]imidazol-2-yl)-I-(4-methoxyphenyl)vinyl4-methoxybenzoate; (Z)-2-(I-isopropyl-5-methyl-IH-benzo[d]imidazol-2-yl)-I-(4-methoxy-phenyl)vinyl-4-methoxybenzoate; 3-alkyl-p-methan-3-ol derivatives; derivatives of fenchyl, D-bomyl, L-bornyl, exo-norbomyl, 2-methylisobomyl, 2-ethylfenchyl, 2-methylbornyl, cis-pinan-2-yl, verbanyl and isobomyl; menthyl oxamate derivatives; menthyl 3-oxocarboxylic acid esters; N-alpha-(Menthanecarbonyl)amino acid amides; p-menthane carboxamide and WS-23 analogs; (-)-(IR,2R,4S)-dihydroumbellulol; p-menthane alkyloxy amides; cyclohexane derivatives; butone derivatives; a mixture of 3-menthoxy-I-propanol and I-menthoxy-2-propanol; I-[2-hydroxyphenyl]-4-[2-nitrophenyl-]-1,2,3,6-tetrahydropyrimidine-2-one; 4-methyl-3-(I-pyrrolidinyl)-2[5H]-furanone; and combinations thereof.

In a particular embodiment, the non-polar phase is present in the flavor delivery system in an amount of from 1 to 50 wt. %, preferably from 20 to 46 wt. %, based on the total amount of the flavor delivery system. In said embodiment, the composition is preferably a microemulsion.

In a particular embodiment, the non-polar phase consists of flavor oil.

In a particular embodiment, flavor oil is present in the flavor delivery system in an amount of at least 1 wt. %, preferably at least 4 wt. %, more preferably at least 10 wt. %, based on the total weight of the flavor delivery system.

By “flavor oil”, it is meant here a flavouring ingredient or a mixture of flavouring ingredients, solvent or adjuvants of current use for the preparation of a flavouring formulation, i.e. a particular mixture of ingredients which is intended to be added to a composition to impart, improve or modify its organoleptic properties, in particular its flavour and/or taste. Taste modulator as also encompassed in said definition. Flavouring ingredients are well known to a skilled person in the art and their nature does not warrant a detailed description here, which in any case would not be exhaustive, the skilled flavourist being able to select them on the basis of his general knowledge and according to the intended use or application and the organoleptic effect it is desired to achieve. Many of these flavouring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, can Nostrand Co., Inc. Solvents and adjuvants or current use for the preparation of a flavouring formulation are also well known in the art.

In a particular embodiment, the flavor oil comprises lime oil. Preferably, the flavor oil consists of lime oil.

In a particular embodiment, the flavor oil comprises grapefruit oil. Preferably, the flavor oil consists of grapefruit oil.

In a particular embodiment, the flavor oil comprises mikan flavor. Preferably, the flavor oil consists of mikan flavor.

The flavor composition further comprises a polar phase comprising at least one polar non-aqueous solvent.

Under “polar phase” is to be understood the total amount of hydrophilic compounds in the flavor delivery system according to the invention.

In a particular embodiment, the polar phase is present in the flavor delivery system in an amount of from 20 to 50 wt. %, based on the total weight of the flavor delivery system, preferably in an amount of from 25 to 40 wt. %.

Under “polar non-aqueous solvent” is to be understood a polar (hydrophilic) solvent that is not water.

In a particular embodiment, the non-aqueous solvent is a food-grade solvent, in particular a non-aqueous solvent for the use for food compositions, in particular in combination with flavor ingredients.

In a particular embodiment, the polar phase comprises a non-aqueous solvent selected from the group consisting of glycerol, propylene glycol, benzylic alcohol, ethanol, propanol, isopropanol, 1,3-propanediol, butanol, butylene glycol, hexylene glycol, dipropylene glycol, ethoxydiglycol, triacetine, or any mixtures thereof. Preferably, the polar non-aqueous solvent is glycerol or propylene glycol, more preferably the polar non-aqueous solvent is propylene glycol.

In a particular embodiment, the polar phase consists of a non-aqueous solvent selected from the group consisting of glycerol, propylene glycol, benzylic alcohol, ethanol, propanol, isopropanol, 1,3-propanediol, butanol, butylene glycol, hexylene glycol, dipropylene glycol, ethoxydiglycol, triacetine, or any mixtures thereof. Preferably, the polar non-aqueous solvent is glycerol or propylene glycol, more preferably the polar non-aqueous solvent is propylene glycol.

In a particular embodiment, the polar phase further comprises water.

The flavor delivery system according to the invention comprises a stabilizer system comprising a biopolymer.

Biopolymers consist of monomeric units that are covalently bound to form larger molecules. Based on the monomers comprised therein, there are three main classes of biopolymers: polynucleotides, polypeptides, and polysaccharides.

In a particular embodiment, the biopolymer is enzymatically or chemically modified.

In a particular embodiment, the biopolymer is present in the flavor composition. In such embodiment, the biopolymer is blended with the flavor composition in the flavor delivery system.

In a particular embodiment, the biopolymer is a polynucleotide, polypeptide, polysaccharide, or any mixture thereof. Preferably, the biopolymer is a polysaccharide.

In a particular embodiment, the biopolymer is modified starch, gum arabic, inulin, maltodextrin, preferably resistant maltodextrin, or mixtures thereof.

In a particular embodiment, the biopolymer is modified starch, inulin, maltodextrin, preferably resistant maltodextrin, or mixtures thereof.

In a particular embodiment, the biopolymer is modified starch or resistant maltodextrin.

In a particular embodiment, the biopolymer is not octenylsuccinic acid-modified gum arabic.

In a particular embodiment, the biopolymer is not a pectin, preferably not a sugar beet pectin.

In a particular embodiment, the biopolymer is present in an amount at least 10 wt. %, preferably at least 20 wt. %, more preferably at least 30 wt. %, based on the total weight of the flavor delivery system.

In a particular embodiment, the weight ratio of the flavor composition to the stabilizer system is from 10:1 to 1:30, preferably from 5:1 to 1:5, more preferably from 2.5:1 to 1:2.5.

In a particular embodiment, the flavor composition further comprises one or more antioxidants. Generally, antioxidants are chemical compounds that inhibit oxidation. Antioxidants can be classified into two groups, depending on whether they are soluble in polar solvents (hydrophilic antioxidants) or in non-polar solvents (lipophilic antioxidants). Examples for hydrophilic antioxidants are ascorbic acid (Vitamin C), green tea extract, water soluble rosemary extract, glutathione, lipoic acid, and uric acid. Examples for hydrophobic antioxidants are carotenes, tocopherols (Vitamin E), butylated hydroxyanisole (BHA), butylated hydroytoluene (BHT), oil soluble rosemary extract and ubiquinol (coenzyme Q). In case the composition is an emulsion, the antioxidant will either be present in the polar or non-polar phase depending on its hydrophilic/lipophilic properties.

In a particular embodiment, the flavor composition further comprises one or more vitamins. A vitamin is an organic molecule (or related set of molecules) that is an essential micronutrient that an organism needs in small quantities for the proper functioning of its metabolism. Similar to what has been said above for antioxidants, vitamins can be classified into two groups, depending on whether they are soluble in polar solvents (hydrophilic vitamins) or in non-polar solvents (lipophilic vitamins). Examples for hydrophilic vitamins are Vitamin C (ascorbic acid) and the B-Vitamins (e.g. B1, B2, B3, B5, B6, B7, and B12). Examples for hydrophobic vitamins are Vitamin A, Vitamin D, Vitamin E, and Vitamin K. In case the composition is an emulsion, the vitamin will either be present in the polar or non-polar phase depending on its hydrophilic/lipophilic properties. In some cases, chemicals compounds are both antioxidants and vitamins, such as Vitamin C (ascorbic acid), Vitamin E (tocopherols). Preferably, the composition comprises Vitamin E and/or Vitamin C as antioxidants.

In a particular embodiment, the flavor composition further comprises one or more sugars. Sugar is the generic name for sweet-tasting carbohydrates that are soluble in polar solvents, such as water. In particular, the term “sugar” refers to monosaccharides, such as glucose, fructose, and galactose, as well as disaccharides, such as sucrose, lactose, and maltose. Longer chains of sugar molecules are not regarded as sugars, and are called oligosaccharides or polysaccharides. Due to their hydrophilic character, sugar compounds are present in the polar phase in case of an emulsion.

In a particular embodiment, the flavor composition further comprises one or more defoamers. A defoamer or an anti-foaming agent is a chemical additive that reduces and hinders the formation of foam, in particular in liquids used for industrial processes. A defoamer can be a water-dispersible defoamer or oil-soluble defoamer. Examples include silicone emulsion antifoamers, polydimethylsiloxane antifoamers, 2-octanol, petrolatum, hop lipids, mineral oil, sorbitan monostearate, and a combination thereof.

In a particular embodiment, the flavor delivery system is clear in appearance. When the flavor delivery system has a NTU value of less than 10, it is considered to have a clear appearance.

The NTU-value refers to “Nephelometric Turbidity Units” that are representative for the turbidity of a composition, and are measured by means of a turbidimeter as specified by the United States Environmental Protection Agency. Preferably, turbidity is measured by a portable turbidity meter (Hanna instruments, Woonsocket, RI, HI93703). Generally, beverages or personal care products having a NTU value above 15 can be considered hazy and not clear. By contrast, beverages and personal care products having a NTU of less than 10 can be considered to have a clear appearance.

Another aspect of the invention relates to a flavor delivery system in the form of a kit-of-parts, wherein the flavor delivery system is a kit-of-parts comprising
a flavor composition comprising
   a surfactant system comprising lysolecithin, lecithin, a sugar ester, a monoglycerol ester, a polyglycerol ester or mixtures thereof,
   a non-polar phase comprising a flavor oil,
   a polar phase comprising at least one polar non-aqueous solvent,
a stabilizer system comprising a biopolymer.

The present invention further relates to a method for preparing a flavor delivery system, wherein the method comprises the steps of:
a. Mixing a non-polar phase comprising a flavor oil and a polar phase comprising at least one polar non-aqueous solvent in the presence of a surfactant system comprising lysolecithin, lecithin, a sugar ester, a monoglycerol ester, a polyglycerol ester or mixtures thereof,
wherein a stabilizer system comprising a biopolymer is added during the mixing step in step a) when forming the flavor composition and/or is added to the flavor composition in a subsequent step b).

According to a particular embodiment, the method comprises the steps of:
a. Mixing a non-polar phase comprising a flavor oil and a polar phase comprising at least one polar non-aqueous solvent in the presence of a surfactant system comprising lysolecithin, lecithin, a sugar ester, a monoglycerol ester, a polyglycerol ester or mixtures thereof, and optionally a stabilizer system comprising a biopolymer to form a flavor composition, and
b. Adding a stabilizer system comprising a biopolymer if not added in step a) to the flavor composition.

According to a particular embodiment, the method comprises the steps of:
a. Mixing a non-polar phase comprising a flavor oil and a polar phase comprising at least one polar non-aqueous solvent in the presence of a surfactant system comprising lysolecithin, lecithin, a sugar ester, a monoglycerol ester, a polyglycerol ester or mixtures thereof, and a stabilizer system comprising a biopolymer to form a flavor composition.

In a particular embodiment, step a) is performed at a temperature from 20 to 25° C.(room temperature).

In a particular embodiment, mixing in step a) of the non-polar phase and the polar phase is performed by using a high-speed homogenizer at between 5000 and 15000 rpm, preferably between 8000 and 12000 rpm, more preferably at 10000 rpm. Preferably, high-speed homogenization is performed for 1 to 10 minutes (min), preferably for 5 minutes (min). Preferably, an IKA, T25 Digital Ultra Turrax®, Germany, is used as high-speed homogenizer.

In a particular embodiment, high-speed homogenization as described above, is followed by a high-pressure homogenization step. Preferably, high-pressure homogenization is performed at 250/50-450/50 bar using a 2-stage high-pressure homogenizer. Preferably, high-pressure homogenization takes place 3 times in a row. Preferably, the high-pressure homogenizer used is a SPXFLOW, APV-1000 lab homogenizer, US. Preferably, a nanoemulsion is obtained after high-pressure homogenization.

In a particular embodiment, the mixing of the non-polar phase and the polar phase in step a) is performed on stir plates at a temperature of from 20 to 25° C.(room temperature).

In a particular embodiment, the adding of a stabilizer system comprising a biopolymer in step b) is before the high-speed homogenization, high-pressure homogenization or stir plate mixing as described above.

The present invention also relates to a beverage or personal care product comprising the flavor delivery system according to the invention.

In a particular embodiment, the beverage or personal care product consists of the flavor delivery system according to the invention.

In a particular embodiment, the beverage or personal care product is acidic. Preferably, the beverage or personal care product has a pH-value of from 2.5 to 7, preferably from 2.6 to 4.0. Due to the combination of the flavor composition and the stabilizer system, the flavor delivery system can also be incorporated in beverages showing low pH-values.

In a particular embodiment, the beverage or personal care product is acidic and has a pH-value of 3.2.

In a particular embodiment, the beverage or personal care product is acidic and has a pH-value of 3.0.

In a particular embodiment, the beverage or personal care product is acidic and has a pH-value of 2.8.

In a particular embodiment, the flavor delivery system is present in an amount of 0.002 to 10 wt. %, preferably from 0.02 to 5 wt. %, based on the total weight of the beverage or personal care product.

In a particular embodiment, the biopolymer is present in an amount from 0.001 to 5 wt. %, preferably from 0.01 to 2 wt. %, based on the total weight of the beverage or personal care composition.

In a particular embodiment, the beverage is an alcoholic or non-alcoholic beverage, preferably the beverage is a non-alcoholic beverage.

In a particular embodiment, the personal care product is a mouthwash. The mouthwash can be an alcoholic or alcohol-free mouthwash.

In a particular embodiment, the beverage or personal care product shows a turbidity (NTU) of less than 10. Preferably, the turbidity (NTU) is between 1 and 7.5.

In a particular embodiment, the beverage or personal care product shows a droplet size of the dispersed phase of between 30 and 150 nm, preferably between 45 and 130 nm. Droplet sizes are preferably measured by a Zetasizer nano ZS (Malvern Panalytical Limited, Worcs, UK). Droplet size of the dispersed phase is preferably between 95 and 125 nm when the composition according to the invention is a microemulsion. Droplet size of the dispersed phase is preferably between 48 and 96 nm when the composition according to the invention is a nanoemulsion.

In a particular embodiment, sugar is present in the beverage or personal care product in an amount of between 2 and 10 wt. %, based on the total weight of the beverage or personal care product, preferably between 3 and 8 wt. %.

In a particular embodiment, citric acid is present in the beverage or personal care product in an amount of between 0.05 and 5 wt. %, based on the total weight of the beverage or personal care product, preferably between 0.1 and 0.5 wt. %.

In a particular embodiment, Vitamin C is present in the beverage or personal care product in an amount of between 0.005 and 1 wt. %, based on the total weight of the beverage or personal care product, preferably between 0.01 and 0.05 wt. %.

In a particular embodiment, trisodium citrate dihydrate and/or sodium citrate is present in the beverage or personal care product in an amount of between 0.005 and 0.5 wt. %, based on the total weight of the beverage or personal care product, preferably between 0.05 and 0.2 wt. %.

The present invention further relates to a method for preparing a flavored beverage or personal care product comprising the step of adding the flavor delivery system according to the invention to a beverage or personal care product or base.

The term “flavored beverage” includes flavored and cream sodas, powdered soft drinks, as well as liquid concentrates such as fountain syrups and cordials; coffee and coffee-based drinks, coffee substitutes and cereal-based beverages; teas, including dry mix products as well as ready-to-drink teas (herbal and tealeaf based); fruit and vegetable juices and juice flavored beverages as well as juice drinks, nectars, concentrates, punches and “ades”; sweetened and flavored waters, both carbonated and still; sport/energy/health drinks; alcoholic beverages plus alcohol-free and other low-alcohol products including beer and malt beverages, cider, and wines (still, sparkling, fortified wines and wine coolers); other beverages processed with heating (infusions, pasteurization, ultra-high temperature, ohmic heating or commercial aseptic sterilization) and hot-filled packaging; and cold-filled products made through filtration or other preservation techniques.

During the preparation of a flavored beverage, the compounds being comprised by the flavor delivery system have to be selected such that they are suitable for human consumption. For example, the polar non-aqueous solvents mentioned above have to be selected such that they are suitable for human consumption. Therefore, butylene glycol and hexylene glycol should not be present in the flavor delivery system.

Personal care products are typically applied to the human body for the purposes of cleaning, beautifying, promoting attractiveness or changing its appearance. Personal care products are for example, toothpaste or mouthwashes.

In a particular embodiment, a mouthwash is prepared. Mouthwashes or mouth rinses are liquid oral care preparations developed to clean and refresh the oral cavity or oral surface by inhibiting or killing the microorganisms that cause malodor, dental caries, tooth decay, gum diseases, gingivitis, and periodontal disorders.

The present invention further relates to the use of a biopolymer for modifying, increasing or enhancing the storage stability of a flavor composition in an acidic beverage or consumer product. Preferably, the biopolymer is modified starch, gum arabic, maltodextrin, or mixtures thereof. In case maltodextrin is used, preferably resistant maltodextrin is used. Preferably, the acidic beverage or consumer product has a pH-value of from 2.5 to 7, more preferably from 2.6 to 4.0.

In a particular embodiment, the acidic beverage or consumer product has a pH-value of 3.2.

Under storage stability, it is understood that the acidic beverage or consumer product shows a NTU-value of below 10 during and upon storage at 40° C. for at least one month, or alternatively, during and upon storage at room temperature for 6-9 months. Under room temperature, temperatures between 20 to 25° C. are understood.

The present invention further relates to a method of modifying, increasing or enhancing the storage stability of a flavor composition in an acidic beverage or consumer product by adding a biopolymer to the acidic beverage or consumer product. Preferably, the acidic beverage or consumer product has a pH-value of from 2.5 to 7, more preferably from 2.6 to 4.0. Preferably, the biopolymer is modified starch, gum arabic, maltodextrin or mixtures thereof. In case maltodextrin is used, preferably resistant maltodextrin is used.

In view of the above, the present invention provides a flavor delivery system that comprises well-established surfactants, but that can nevertheless be used for the preparation of acidic beverages or personal care products.

In particular, the inventive flavor delivery system can be conveniently used for the preparation of an acidic beverage or personal care product that is clear in appearance and that shows stability during prolonged storage.

### EXAMPLE

### 1. Preparation of Flavor Microemulsions

Flavor microemulsions according to Samples A to E were prepared by mixing all the ingredients of each sample as shown in Table 1 on magnetic stir plates at room temperature.

TABLE 1
Flavor microemulsion compositions.

| | Sample | | | | |
|---|---|---|---|---|---|
| | A | B | C | D | E |
| Ingredient | % wt | % wt | % wt | % wt | % wt |
| Lime oil | 45.5 | — | 35 | — | 20 |
| Grapefruit oil | — | 36 | — | — | |
| Mikan flavor | — | — | — | 35 | |
| S LPC80 | 19.6 | 21 | 28 | 28 | |
| LIPOID P 20 | | | | | 0.6 |
| LIPOID P 45 | 4.9 | — | — | — | |
| LIPOID H 100 | — | 3 | — | — | 1.2 |
| F160 | — | — | 7 | 7 | |
| Sugar ester P90 | | | | | 12 |
| Propylene glycol | 30 | 40 | 30 | 30 | 25 |
| Glycerol | | | | | 12.5 |
| Butanol | | | | | 0.5 |
| Fructose | | | | | 15 |
| Water | | | | | 13.2 |
| Total | 100 | 100 | 100 | 100 | 100 |

Further details as to ingredients mentioned above are given in Table 2 below:

Ingredient

| | |
|---|---|
| S LPC80 | Lysolecithin product containing at least 80 wt. % |
| | of lysolecithin (ex. Lipoid GmbH) |
| LIPOID H 100 | Sunflower lecithin containing at least 90 wt. % of |
| | phosphatidylcholine (ex. Lipoid GmbH) |
| LIPOID P 45 | Fat free soybean lecithin with 45% phosphatidylcholine from non- |
| | genetically-modified plants (ex. Lipoid GmbH) |
| LIPOID P 20 | Fatfree soybean lecithin with 20% phosphatidylcholine (ex. Lipoid GmbH) |
| F160 | Sugar ester containing 70% sucrose monostearate (ex. DK ESTER) |
| Sugar ester P90 | Sugar ester containing at least 90% of sucrose |
| | monopalmitate (ex. Compass Foods) |

### 2. Preparation of Beverages Using the Flavor Microemulsions According to Samples A to E

Beverages (pH-value of 3.2) comprising flavor microemulsions A (A1-A4) and B (B1-B4), respectively, were prepared according to Table 3. The beverages with or without added biopolymers (modified starch or resistant maltodextrin) were pasteurized at 92° C. for 1 min and stored at 40° C. for one month. The appearances of the beverages were recorded after storage. The turbidity of the beverages was measured by a portable turbidity meter (Hanna instruments, HI93703, Woonsocket, RI) and was reported in Nephelometric Turbidity Unit (NTU).

TABLE 3
Final beverage compositions (beverages A1-A4, and beverages B1-B4)

| | A1 | A2 | A3 | A4 | B1 | B2 | B3 | B4 |
|---|---|---|---|---|---|---|---|---|
| | Mass | Mass | Mass | Mass | Mass | Mass | Mass | Mass |
| Ingredients | (g) | (g) | (g) | (g) | (g) | (g) | (g) | (g) |
| Flavor | 0.088 | 0.088 | 0.088 | 0.088 | — | — | — | — |
| composition A | | | | | | | | |
| Flavor | — | — | — | — | 0.11 | 0.11 | 0.11 | 0.11 |
| composition B | | | | | | | | |
| Sugar | 60 | 60 | 60 | 60 | 60 | 60 | 60 | 60 |
| Citric Acid | 3 | 3 | 3 | 3 | 3 | 3 | 3 | 3 |
| Vitamin C | 0.2 | 0.2 | 0.2 | 0.2 | 0.2 | 0.2 | 0.2 | 0.2 |
| Sodium Citrate | 1 | 1 | 1 | 1 | 1 | 1 | 1 | 1 |
| Modified starch | — | 0.1 | 0.25 | — | — | 0.1 | 0.25 | — |
| Resistant | — | — | — | 1 | — | — | — | 1 |
| maltodextrin | | | | | | | | |
| Water up to | 1000 g | 1000 g | 1000 g | 1000 g | 1000 g | 1000 g | 1000 g | 1000 g |
| Appearance* | x | ∘ | ∘ | ∘ | x | ∘ | ∘ | ∘ |
| NTU | — | 5.69 | 3.36 | 7.29 | — | 1.34 | 4.76 | 2.51 |

*x: precipitated (not clear); ∘: clear beverage

Likewise, beverages (pH-value of 3.2) comprising flavor microemulsions C (C1-C4) and D (D1-D4), respectively, were prepared according to Table 4, pasteurized at 92° C. for 1 min, and stored at 40° C. for one month. The appearances of the beverages were recorded after storage.

TABLE 4
Final beverage compositions (beverages C1-C4, and beverages D1-D4)

| | C1 | C2 | C3 | C4 | D1 | D2 | D3 | D4 |
|---|---|---|---|---|---|---|---|---|
| | Mass | Mass | Mass | Mass | Mass | Mass | Mass | Mass |
| Ingredients | (g) | (g) | (g) | (g) | (g) | (g) | (g) | (g) |
| Flavor | 0.11 | 0.11 | 0.11 | 0.11 | — | — | — | — |
| composition C | | | | | | | | |
| Flavor | — | — | — | — | 0.11 | 0.11 | 0.11 | 0.11 |
| composition D | | | | | | | | |
| Sugar | 60 | 60 | 60 | 60 | 60 | 60 | 60 | 60 |
| Citric Acid | 3 | 3 | 3 | 3 | 3 | 3 | 3 | 3 |
| Vitamin C | 0.2 | 0.2 | 0.2 | 0.2 | 0.2 | 0.2 | 0.2 | 0.2 |
| Sodium | 1 | 1 | 1 | 1 | 1 | 1 | 1 | 1 |
| Citrate | | | | | | | | |
| — | — | 0.1 | 0.25 | — | — | 0.1 | 0.25 | — |
| starch | | | | | | | | |
| Resistant | — | — | — | 1 | — | — | — | 1 |
| maltodextrin | | | | | | | | |
| Water up to | 1000 g | 1000 g | 1000 g | 1000 g | 1000 g | 1000 g | 1000 g | 1000 g |
| Appearance* | x | ∘ | ∘ | ∘ | x | ∘ | ∘ | ∘ |
| NTU | — | 2.11 | 2.37 | 6.38 | — | 2.23 | 1.33 | 5.98 |

*x: precipitated (not clear); ∘: clear beverage

In addition, beverages (pH-value of 2.8) comprising flavor microemulsions E (E1-E2) and flavor microemulsions A5 and A6 were prepared according to Table 5, pasteurized at 92° C. for 1 min, and stored at room temperature for 9 months for E1-E2, and 8 months for A5-A6, respectively.

TABLE 5
Final beverage compositions (beverages E1-E2)

| | E1 | E2 | A5 | A6 |
|---|---|---|---|---|
| | Mass | Mass | Mass | Mass |
| Ingredients | (g) | (g) | (g) | (g) |
| Flavor | 0.11 | 0.11 | — | — |
| composition E | | | | |
| Flavor | — | — | 0.088 | 0.088 |
| composition A | | | | |
| Sugar | 60 | 60 | 60 | 60 |
| Citric Acid | 3 | 3 | 3 | 3 |
| Vitamin C | 0.2 | 0.2 | 0.2 | 0.2 |
| Sodium | 0.5 | 0.5 | 0.5 | 0.5 |
| Citrate | | | | |
| Modified | — | 0.1 | — | — |
| starch | | | | |
| Inulin | — | — | — | 0.5 |
| Water up to | 1000 g | 1000 g | 1000 g | 1000 g |
| Appearance* | x | ∘ | x | ∘ |

*x: precipitated (not clear);
∘: clear beverage

As can be seen from Tables 3, 4 and 5, the addition of a biopolymer such as modified starch, resistant maltodextrin, or inulin significantly improved the stability of the emulsified flavor composition in the acidic beverages. In particular, after one-month of storage at a high temperature (40° C.) or 8-9 months of storage at room temperature, beverages containing a biopolymer were still clear in appearance, and their NTU-value was less than 10, whereas beverages without added biopolymers showed precipitation and were not clear in appearance upon storage.

1. A flavor delivery system comprising
a flavor composition comprising
a surfactant system comprising lysolecithin, lecithin, a sugar ester, a monoglycerol ester, a polyglycerol ester, or mixtures thereof,
a non-polar phase comprising a flavor oil,
a polar phase comprising at least one polar non-aqueous solvent;
wherein, the flavor delivery system includes a stabilizer system comprising a biopolymer.

2. The flavor delivery system according to claim 1, wherein the flavor delivery system is in form of a microemulsion or nanoemulsion.

3. The flavor delivery system according to claim 1, wherein the flavor oil is present in an amount of at least 1 wt. %, based on the total weight of the flavor delivery system.

4. The flavor delivery system according to claim 1, wherein the biopolymer is modified starch, gum arabic, inulin, maltodextrin, or mixtures thereof.

5. The flavor delivery system according to claim 1, wherein the biopolymer is present in an amount at least 10 wt. %, based on the total weight of the flavor delivery system.

6. The flavor delivery system according to claim 1, wherein the weight ratio of the flavor composition to the stabilizer system is from 10:1 to 1:30.

7. The flavor delivery system according to claim 1, wherein the polar phase further comprises water.

8. A method for preparing a flavor delivery system, wherein the method comprises the steps of:
a) Mixing a non-polar phase comprising a flavor oil and a polar phase comprising at least one polar non-aqueous solvent in the presence of a surfactant system comprising lysolecithin, lecithin, a sugar ester, a monoglycerol ester, a polyglycerol ester or mixtures thereof,
wherein a stabilizer system comprising a biopolymer is added during the mixing step in step a) when forming the flavor composition and/or is added to the flavor composition in a subsequent step b).

9. A beverage or personal care product comprising a flavor delivery system according to claim 1.

10. The beverage or personal care product according to claim 9, wherein the beverage or personal care product is acidic.

11. The beverage or personal care product according to claim 9, wherein the flavor delivery system is present in an amount of 0.002 to 10 wt. %, based on the total weight of the beverage or personal care product.

12. The beverage or personal care product according to claim 9, wherein the biopolymer is present in an amount from 0.001 to 5 wt. %, based on the total weight of the beverage or personal care composition.

13. A method for preparing a flavored beverage or personal care product comprising the step of adding the flavor delivery system according to claim 1 to a beverage or personal care product or base.

14. (canceled)

15. Method of modifying, increasing or enhancing the storage stability of a flavor composition in an acidic beverage or consumer product, by adding a biopolymer, to the acidic beverage or consumer product.

16. The flavor delivery system according to claim 4, wherein the biopolymer is resistant maltodextrin.

17. The flavor delivery system according to claim 5, wherein the biopolymer is present in an amount of at least 20 wt. %, based on the total weight of the flavor delivery system.

18. The flavor delivery system according to claim 6, wherein the weight ratio of the flavor composition to the stabilizer system is from 5:1 to 1:5.

19. The flavor delivery system according to claim 18, wherein the weight ratio of the flavor composition to the stabilizer system is from 2.5:1 to 1:2.5.

20. The beverage or personal care product according to claim 10, wherein the beverage or personal care product has a pH-value of from 2.5 to 7.

21. The beverage or personal care product according to claim 20, wherein the beverage or personal care product has a pH-value of from 2.6 to 4.0.
